(19) United States
(12) Patent Application Publication
(10) Pub. No.: US 2020/0082939 A1
(43) Pub. Date: Mar. 12, 2020

(54) EVALUATION SYSTEM AND METHOD OF USE THEREOF

(71) Applicant: David A. Dill, Newtown, PA (US)

(72) Inventor: David A. Dill, Newtown, PA (US)

(21) Appl. No.: 16/124,750

(22) Filed: Sep. 7, 2018

(51) Int. Cl. G16H 50/20; G06F 17/30; G16H 15/00; G06F 15/18

(52) U.S. Cl. CPC G16H 50/20; G06F 15/18; G16H 15/00; G06F 17/3089

(57) ABSTRACT

A method for monitoring and modifying behavior to reduce negative effects, including the steps of receiving, via a user interface of an application executing on one or more computer processors, a negative information report about one or more discriminatory acts or offensive behaviors known or alleged to have been committed by an individual or entity; assigning, via the one or more computer processors, a negative information point value for any of a plurality of discriminatory acts or offensive behaviors and a discriminatory/offensive behaviors point matrix stored on a memory device accessible by the one or more computer processors, the discriminatory/offensive behaviors point matrix being generated and continually updated by a machine learning module; storing the negative information report on a negative information database; notifying the individual or entity about the negative information report; and providing a website for reporting negative information reports under certain specified conditions.

### FIELD

The present application relates generally to a system and method for monitoring and improving behavior and reducing negative behavioral effects.

### BACKGROUND

The prejudicial and/or unjust treatment of different categories of people or things, especially on the grounds of race, age or sex, has deleterious consequences on our society, from an economic, financial and/or social perspective. For example, behaviors or actions that negatively impact members of a particular racial or ethnic group may reduce their access to economic opportunities, which may lead to poverty. Such “negative” behaviors may be reflection of, for example, one's prejudices, bias, bigotry, intolerance, or unfair treatment of others. It is difficult to quantify the personal harm or financial burdens incurred by those who are affected by negative behaviors, such as discrimination based on gender, race, age, religion or physical disabilities and the like. Further, it is difficult for policy makers or members of groups thought to be free from certain negative behaviors to appreciate consequences of such negative behaviors. In some cases, individuals may be negatively impacted by behaviors adversely targeting a plurality of attributes in a given individual, including discrimination based on their gender, race, age and/or sexual orientation.

Currently, there are not sufficient systems in place to effectively deal with this problem. Indeed, policy-makers and private sector organizations typically lack effective tools to objectively assess their own deficiencies and address the problems. Accordingly, there is a need for systems and methods to monitor improper discrimination practices and provide a means for addressing these practices so as to reduce the negative consequences resulting therefrom.

### SUMMARY

One aspect of the present application relates to a method for monitoring and modifying behavior to reduce negative effects, comprising the steps of: (a) receiving, via a user interface of an application executing on one or more computer processors, a negative information report about one or more discriminatory acts or offensive behaviors known or alleged to have been committed by an individual or entity, said information being obtained from a private and/or confidential source, a website reporting said discriminatory acts or offensive behaviors news, or both; (b) assigning, via the one or more computer processors, a negative information point value for any of a plurality of discriminatory acts or offensive behaviors and a discriminatory/offensive behaviors point matrix stored on a memory device accessible by the one or more computer processors, the discriminatory/offensive behaviors point matrix being generated and continually updated by a machine learning module; (c) determining, via the one or more computer processors, a total negative information point value of the individual or entity via the one or more computer processors, based on the sum total of negative information point values in real-time; (d) storing the negative information report on a negative information database; (e) notifying the individual or entity about the negative information report; and (f) providing a website for reporting negative information reports.

In some embodiment, the negative information report is based on information obtained from a private and/or confidential source. In other embodiments, the negative information report is based on information obtained from an online news source.

In one embodiment, the machine learning module comprises a search engine that automatically tracks reported incidences of discriminatory or offensive behaviors by the individual or entity in one or more websites. In another embodiment, the machine learning module comprises a search engine that monitors the incidences of discriminatory or offensive behaviors using search terms or phrases consistent with the exercise of discriminatory or offensive behaviors. In another embodiment, the machine learning module comprises a learning tool for identifying effective strategies for addressing discriminatory or offensive behaviors, based on effective response strategies identified and processed by the machine learning module.

In some embodiments, the method comprises the step of verifying the authenticity of the negative information report and/or listing the negative information report on the website in step (f). In other embodiments, the negative information report is listed on the website in step (f) only after verifying the authenticity of the negative information report.

In some embodiments, the step of notification further comprises notifying the individual or entity of a proposed response strategy to address one or more discriminatory acts or offensive behaviors alleged to have been committed by the individual or entity, wherein the proposed response is determined by the machine learning module. In other embodiments, the negative information report is withheld from being listed on the website in step (f) when the individual or entity has completed the proposed response strategy within a specified time period.

In some embodiments, the method further comprises the step of listing in the website positive actions taken by the individual or entity with respect to one or more negative information reports. Positive actions include, but are not limited to, the hiring of minorities or special events for various minorities.

In another embodiment, the method further comprises the step of tracking over time, via the one or more computer processors and the total negative information point values, the relative improvement or lack of improvement over time with respect to discriminatory acts or offensive behaviors by the individual or entity.

In another aspect, a system is provided comprising: one or more computer processors; and one or more tangible computer readable media accessible by the one or more computer processors, wherein the one or more tangible computer readable media comprise instructions that, when executed by the one or more processors, cause the one or more processors to perform the above-described steps.

In another embodiment, the system further comprises: (i) one or more computing devices in data communication with each other, each device having one or more computer processors, a data communication connection, and one or more tangible non-transitory computer-readable media accessible by the one or more computer processors, and (ii) a plurality of databases, including a verification database, a negative information database, and a behavioral incentive database, wherein the verification database, negative information database and behavioral incentive database are each stored in the one or more tangible non-transitory computer-readable media.

In a particular embodiment, behavioral incentive database comprises information regarding behavioral incentives and information regarding restorative justice. The information may include, but is not limited to, shared information on the pain caused by discrimination, even if it is subtle and might not even be intentional.

In another embodiment, the plurality of databases further comprises a restorative justice database.

In a further aspect, the present application provides a tangible non-transitory computer readable storage medium, comprising instructions that, when executed by a computer processor, cause the processor to: (a) receive, via a user interface of an application executing on one or more computer processors, a negative information report about one or more discriminatory acts or offensive behaviors known or alleged to have been committed by an individual or entity, said information being obtained from a private and/or confidential source, a website reporting said discriminatory acts or offensive behaviors news, or both; (b) assign, via the one or more computer processors, a negative information point value for any of a plurality of discriminatory acts or offensive behaviors and a discriminatory/offensive behaviors point matrix stored on a memory device accessible by the one or more computer processors, the discriminatory/offensive behaviors point matrix being generated and continually updated by a machine learning module; (c) determine, via the one or more computer processors, a total negative information point value of the individual or entity via the one or more computer processors, based on the sum total of negative information point values in real-time; (d) store the negative information report on a negative information database; (e) notify the individual or entity about the negative information report, and (f) list the negative information report on a website when certain conditions are met.

### BRIEF DESCRIPTION OF THE DRAWINGS

The present invention can be better understood by reference to the following drawings, wherein like references numerals represent like elements. The drawings are merely exemplary to illustrate certain features that may be used singularly or in any combination with other features and the present invention should not be limited to the embodiments shown.

FIG. 1 is a flow chart showing an exemplary sequence of steps of the method of the present application.

FIG. 2 shows an exemplary system the present of the present application.

FIG. 3 shows a more detailed view of an exemplary system the present application.

### DETAILED DESCRIPTION

The following detailed description is presented to enable any person skilled in the art to make and use the object of this application. For purposes of explanation, specific nomenclature is set forth to provide a thorough understanding of the present application. However, it will be apparent to one skilled in the art that these specific details are not required to practice the subject of this application. Descriptions of specific applications are provided only as representative examples. The present application is not intended to be limited to the embodiments shown, but is to be accorded the widest possible scope consistent with the principles and features disclosed herein.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

As used in this application, the terms “component”, “module”, “system”, “interface”, and the like are generally intended to refer to a computer-related entity, either hardware, a combination of hardware and software, software, or software in execution. For example, a component may be, but is not limited to being, a process running on a processor, a processor, an object, an executable, a thread of execution, a program, or a computer. By way of illustration, both an application running on a controller and the controller can be a component. One or more components residing within a process or thread of execution and a component may be localized on one computer or distributed between two or more computers. As used herein, the term “wireless” means any wireless signal, data, communication, or other interface including without limitation Wi-Fi, Bluetooth, 3G, 4G, HSDPA/HSUPA, TDMA, CDMA (e.g., IS-95A, WCDMA, etc.), FHSS, DSSS, GSM, PAN/802.15, Wi-MAX (802.16), 802.20, narrowband/FDMA, OFDM, PCS/DCS, analog cellular, CDPD, satellite systems, millimeter wave or microwave systems, acoustic, and infrared (i.e., IrDA).

As used herein, the terms “Internet” and “internet” are used interchangeably to refer to inter-networks including, without limitation, the Internet.

As used herein, the term “memory” includes any type of integrated circuit or other storage device adapted for storing digital data including, without limitation, ROM. PROM, EEPROM, DRAM, SDRAM, DDR/2 SDRAM, EDO/FPMS, RLDRAM, SRAM, “flash” memory (e.g., NAND/NOR), and PSRAM.

As used herein, the term “computer processor” refers generally to all types of digital processing devices including, without limitation, digital signal processors (DSPs), reduced instruction set computers (RISC), general-purpose (CISC) processors, microprocessors, gate arrays (e.g., FPGAs), PLDs, reconfigurable compute fabrics (RCFs), array processors, and application-specific integrated circuits (ASICs). Such digital processors may be contained on a single unitary IC die or distributed across multiple components.

The flowchart and block diagrams in the figures illustrate the architecture, functionality, operation, and/or implementation of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may be executed in the reverse order, depending upon the functionality involved. In addition, each block in a block or combination of blocks in a diagram and/or flowchart illustration can be implemented by special purpose hardware-based systems that perform the specified functions, acts or combinations thereof may be carried out using special purpose hardware and/or computer instructions.

### Methods for Monitoring and Reducing Negative Behaviors

One aspect of the present application relates to an Internet-based method for monitoring and reducing “negative behaviors” among individuals and entities. As used herein, the phrase “negative behaviors” relates to improper discriminatory behaviors and/or offensive behaviors. In particular, the method monitors the incidence of these socially unacceptable behaviors in the market place so as to highlight an unsupportive or hostile consumer experience characterized by one or more forms of improper discriminatory and/or offensive behaviors practiced by an individual or an organization or business. Examples of improper discriminatory behaviors, include, but are not limited to discrimination based on gender, race, age, ethnicity, disability, marital status, sexual orientation, political affiliation, national/geographic origin, religion, language, immigration status, medical status (e.g., HIV status), pregnancy status, criminal record, personal appearance (e.g., weight, dress), social class, address or zip code, geographical origin, and economic status (e.g., poverty, unemployment, stigmatized jobs (e.g. sex work)). As used herein, the term “discrimination” is intended to be used broadly with respect to any behavior(s) or action(s) associated with differential treatment of individuals according to any of the foregoing status groups.

Examples of offensive behaviors include, but are not limited to rude behavior, obscene language, racially offensive language, sexually offensive language (e.g., jokes, stories, suggestive language), threats, false rumors, physical abuse, sexual abuse, or any other behaviors contributing to a hostile work or retail shopping or other environment.

In some embodiments, the information/data sources utilized for monitoring these negative behaviors include a negative information database comprising negative information reports directly submitted by individuals directly witnessing discriminatory acts or offensive behaviors. Alternatively, or in addition, the information/data sources include third-party news organization websites, social media applications (e.g., Facebook, Twitter, Instagram etc.), and legal database sources (e.g., reporting lawsuits against individuals or entities).

In some embodiments, the desired data or information may be identified on the basis of one or more of keywords, tags, rules, or other suitable method or process. The identified data or information may then be processed using one or more data analysis/decision making techniques.

In certain embodiments, a machine learning module employing machine learning (either supervised or unsupervised) techniques is applied to the data mined and processed in order to formulate more effective response strategies, based on those which have been shown to be effective in similar instances.

The monitoring is designed to inform the public regarding the extent to which individuals or entities have engaged in discriminatory and/or offensive behaviors, and further inform the individual or entities themselves, who may not otherwise recognize the negative behaviors displayed or their potential impact on others. This can facilitate appropriate remedial actions by the individuals or entities, such as those prescribed in accordance with the systems herein as further described below. Thus, the method can be used to inform individuals or entities who are known or alleged to have exercised, wittingly or unwittingly, discriminatory or offensive behaviors so that steps can be taken to reverse the negative behaviors and actions and to further inform the public regarding improvements and steps taken by the individuals or entities to address the previous deficiencies. This can allow consumers to make better informed decisions about whether to financially support these individuals or entities.

The negative information database may further include reports describing positive steps taken to address the negative behaviors. In this case, the positive steps may be used in conjunction with the machine learning module to reduce the real-time discriminatory/offensive behavior scores obtained from the behavioral point matrix.

In some embodiments, individual(s) or entities may be informed about discriminatory or offensive behaviors by the issuance of one or more alerts. The alerts may be contained in an alert database. Alternatively, a user may search the system or alert database to identify and evaluate the scope of alerts attributed to a given individual or entity.

In certain embodiments, the method further comprises the steps of: retrieving, via the one or more computer processors, information about improper discriminatory and/or offensive behavior, and electronically delivering, via the one or more computer processors, an alert to an affected individual or entity over a wireless communication channel to a wireless device associated with the individual or entity, wherein the alert activates an application on the wireless device that causes the wireless device to connect, via the Internet, to the one or more computer processors and download the information.

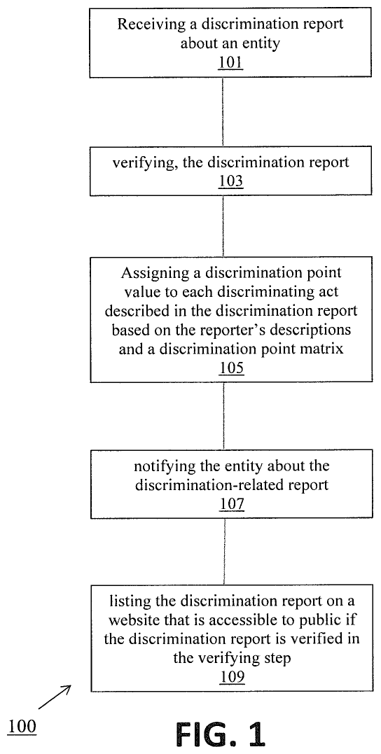

As shown in FIG. 1, one embodiment of the method 100 comprises the steps of receiving (101), via a user interface of an application executing on one or more computer processors, a negative information report about an entity, verifying (103), via the one or more computer processors, the negative information report, assigning (105), via the one or more computer processors, a negative information point value to each negative behavioral act described in the negative information report based on the reporter's descriptions and a behavior point matrix stored on a memory device accessible by the one or more computer processors; notifying (107) the entity about the negative information report, and listing (109) the negative information report on a website that is accessible to public if the negative information report is verified in the verifying step. The method is implemented by an Internet based application at the application's website.

In some embodiments, the method provides a system comprising: (i) one or more computing devices in data communication with each other, each device having one or more computer processors, a data communication connection, and one or more tangible non-transitory computer-readable media accessible by the one or more computer processors, and (ii) a plurality of databases, including a verification database, a negative information database, and a behavioral incentive database, wherein the verification database, negative information database and behavioral incentive database are each stored in the one or more tangible non-transitory computer-readable media.

In some embodiments, the one or more tangible non-transitory computer readable media further comprise instructions that, when executed by the one or more processors, cause the one or more processors to perform the step of transmitting an alert of a negative behavioral report to the affected individual or entity. This can inform the individual or entity of problems that need to be addressed in order to maintain a good standing or reputation in the public domain.

In some embodiments, the negative information report contains a personal profile and an event profile. The personal profile comprises personal data about the reporter. The personal data may include one or more of the following: name, gender, age, home and/or work address, e-mail address and phone number. The event profile comprises descriptions of a negative behavior or act alleged to have been committed by an individual or entity. As used hereinafter, the “entity” may be an organization, a company, an institution, or a group of individuals. Examples of entities include, but are not limited to, restaurants, hotels, shops, supermarkets, movie theaters, health clubs, phone companies, utility companies, airlines, train lines, bus lines, airports, hospitals, schools, churches, doctor's offices, law firms, lobbying firms, accounting firms, factories, office buildings, corporations, partnerships, colleges and universities. In some embodiments, the entity is a group of people, such as a youth group, a ladies' club, a men's club, a sports team, a police force, and the like.

The negative behavior or act may be any behavior or act that may be perceived as an act of improper discrimination or offensive behavior, such as displayed racist behaviors or attitudes or prejudices or other offensive behavior.

In some embodiments, the method requires user registration and verifies the identity of a registered user by a third party. In some embodiments, only registered users of the application can make a negative information report. In some embodiments, the method allows both registered and unregistered users to generate negative information reports. In some embodiments, the report further includes audio, pictures and/or video information demonstrating behavior by the entity, including where possible the names of individuals causing the problems identified.

In some embodiments, the negative information report is verified by verifying the reporter's personal profile. In some embodiments, the reporter's personal profile passes the verification if 2, 3 or 4 items, such as name, home address, phone number and/or e-mail address, in the reporter's personal profile are confirmed. In some embodiments, the verifying step includes the substep of analyzing accumulated data to determine the credibility of those who reported the discriminatory or offensive act (e.g., to avoid having a store's competitor provide distorting results). In some embodiments, the verifying step includes cross checking reports by accredited individuals who may be assigned by one or more recognized authorities to test the entity's behavior. Input from such accredited individuals could be used to clarify any disputed behavior that may have been reported by random individuals. This would be especially important if reports are alleged to be misleading or fraudulent by entities fearing their competitors might be providing false reports. In addition, the systems described herein are able to recognize the use of mass reporting either by organized groups of individuals or by machine-based algorithms to bias the system against particular entities or individuals.

In some embodiments, the identity of the reporting individual is kept anonymous by maintaining a separate secured database of the identity of such individuals. In particular embodiments, anonymous reports may be submitted, but are then compared to public reports to see if there are consistencies in the reports. In other embodiments, the number of anonymous reports is assessed with verification that each anonymous report appears to have come from separate individual reporters (e.g. different IP addresses) confirmed by the system. In other embodiments, two-step verification (as typically used by sending a text to a mobile phone of the user) of users is required to ensure that users are who they say they are. The value of anonymous reports may be of greater or lesser significance based on the negative behavior that is reported. For example, if the negative behavior that is reported is considered relatively minor then an anonymous report may be given lesser weight. However, if a negative behavior is considered significantly negative in effect on an individual then an anonymous report may be given greater weight.

For example, if the victim of a negative behavior may be societally stigmatized for being a victim then such a report may be anonymous due to the victim's fear of societal stigma. The methods and systems described herein may then ascribe a high point value to this anonymous report because, even if the identity of the reporter cannot be identified, the significance that such report exists is sufficient to justify informing an entity of the anonymous report in a negative information report.

In some embodiments, the method further comprises the steps of assigning the reported act to a defined category of negative behavior (i.e., racism, sexism, anti-gay, etc.) and determining, via the one or more computer processors, a negative information point value for reported negative behavioral or acts based on a behavior point matrix;

The behavior point matrix is generated, and is updated regularly, by evaluating a large-scale data base reflecting discriminatory acts or offensive behaviors described by other reporters, as well as discriminatory acts or offensive behaviors described in other sources, e.g., newspapers, internet, blogs, twitter, etc. In some embodiments, the behavior point matrix contains a list of behavioral context categories (e.g., lodging, food, entertainment, medical/dental, legal, etc.). Each category further contains a list of negative acts (e.g., verbal abuse, physical abuse, material abuse etc.). A point value is assigned to each negative behavioral act in each behavioral context category. In some embodiments, several point values are assigned to each negative behavior/act in each behavior context category based on the severity level of the negative behavior/act.

In some embodiments, the matrix is generated using machine learning and a large database of negative behavioral acts and related events. In some embodiments, the database is being analyzed by a machine learning module for many types of information such as verbal expression patterns, sentence structure, key words, gestures, body movement, environment, personal information (e.g., race, gender, religion, age, etc.) of the person exercising the discriminatory act or offensive behavior, personal information of the person being discriminated against, etc. The module may contain one or more memory units recording several types of information. Examples of the types are the patterns which express the information, connective information with the other memory unit expressing relations between the information (the number of connections, address of the connection, a relation with the patterns to connect), conditions to activate (compare and match conditions), results of the analysis (sources of the information, reliability, newness, fields, theme, types of the sentences, etc.).

Examples of sentence structure include normal sentences, interrogative sentences, imperative sentences, conditional sentences, exclamatory sentences, the truth, facts, rules, common sense, definitions, logic, explanations, hypotheses, predictions, opinions, impressions, rumors, conversion to the absolute words, numerical expressions, physical expressions and chemical expressions and symbols, etc. They are analyzed and recorded in the memory units.

When the information is input, a field of the information, a theme of the information, a type of a sentence, a structure (subjects, predicates, modifiers of the subjects, modifiers of the predicates, relations of the modifiers, the types of the sentences, numerical expressions, physical expressions, chemical expressions, symbols, when, where, who, what, how, why) is analyzed, and the behavioral value of the information is evaluated by comparing with the information recorded in the memory units.

In some embodiments, the machine learning module further analyzes relations among the information. There are various types of relations among the information, such as, cause and result, phenomenon and reason, explanation and result, outline and detail, similar meanings, opposite meanings, main body and summary, main body and relational information etc. These relations are set by the human instructions or set autonomously by learning the relations between information. Based on the above described analysis, a negative behavior point or behavioral severity points are assigned to each behavioral act. The points are adjusted automatically when more data becomes available to the machine learning module.

In some embodiments, the machine learning module comprises a search engine. This search engine performs Internet-wide searches based on lists of words or phrases that are banned by major social media providers or other major social networks. The frequency and consistency with which particular words or phrases are banned from usage on these third party websites is scored by the search engine. The machine learning then calculates a point value that quantifies the level of negative behavior attached to the use of the particular word or phrase based on the frequency with which that word or phrase is banned. In some embodiments, the search engine can search for the frequency of banning for users of social media or other websites with large scale public comment sections for the use of particular words or phrases. The machine learning can then combine the frequency with which users are banned for particular negative behaviors on social media and other websites with the frequency with which particular words or phrases are banned. The machine learning can then arrive at a further point ranking of the degree of negative behavior that is socially attached to the use of particular words or phrases.

Based on changes in the frequency of banning or the usage of particular phrases, the machine learning can learn over time how society adjusts its understanding of the negativity associated with the certain behaviors or use of particular words and phrases. In particular, since many websites may use human moderation to ban users for negative behavior rather than only word filters, the machine learning will be able to identify evolving standards of behavior as they are revealed by the preferences of major social media sites or other websites. The search engine results can be analyzed by the machine learning for such comparative information as a theme of the information, a type of a sentence, a structure (subjects, predicates, modifiers of the subjects, modifiers of the predicates, relations of the modifiers, the types of the sentences, numerical expressions, physical expressions, chemical expressions, symbols, when, where, who, what, how, why), etc. The machine learning can than calculate based on the frequency with which certain corresponding behaviors are banned on one or more sites the extent to which such behaviors have become seen as negative by a wide cross-section of society.

In certain embodiments, the search engine can also monitor the use of particular words and phrases on websites that have been designated hate sites. Hate sites are websites that embrace negative behavior and seek to promote particular negative behavior, e.g., racism. By monitoring the frequency of certain terms on hate sites the machine learning will learn to recognize when certain coded words or phrases may have been adopted by promoters of negative behavior. The machine learning can then assign a point value to such words and phrases that will rank their potential offensiveness alongside more commonly recognized negative behavior. This enables the machine learning to recognize when promoters of negative behavior may be using major social media websites, or other heavily trafficked websites that particular businesses may own, as a means of promoting negative behavior. The machine learning can then ensure that this information is input into the negative information report given to a business with, for example, a public-facing heavily trafficked website.

In certain embodiments, the machine learning can also monitor media style guides, university speech codes, corporate speech policies, and other forms of language guide produced by organizations representing minorities or other groups that experience the consequences of negative behavior. By monitoring university speech codes or corporate speech codes, the machine learning can learn evolving standards of behavior and through the application of the methods and systems described herein ensure wider dissemination of best practices. In particular embodiments, the search engine can monitor the incidence of media reports of certain negative behaviors and score the frequency of comments that such reports then receive on the Internet as a means to assessing the social significance of a particular negative behavior.

Consequently, in certain embodiments, the behavior point matrix comprises point values that reflect information derived from point scores of the negative behavioral value of particular behaviors identified and determined from information gathered from multiple sources across the Internet. The behavior point matrix can evolve over time in its scoring of particular negative behaviors, including the introduction to the matrix of new negative behaviors that involve the use of new terminology or particular negative acts. The behavior point matrix can also have particular negative behaviors manually entered with significant scores by users, although the machine learning does not require such input to assess and assign a point value to particular negative behaviors.

The search engine is also able to search for the use of particular words or phrases that have been identified as negative behavior in contexts where no banning has occurred, or the terms that do not appear frequently on lists of banned words. This may reflect that the particular word or phrase has both a negative meaning and a positive meaning which is dependent on context. The machine learning can assess the context of the use of a word or phrase to determine when that word or phrase is being used negatively. For example, the machine learning may determine that a particular word may be used both positively with respect to sexual orientation, and yet the same word can also be used negatively by some groups as a means of insult. The machine learning has the capability of assessing sentence structure, grammar and phrasing to distinguish between the different contexts of use for a word or phrase.

Accordingly, the behavior point matrix represents a sophisticated filter regarding the usage of word or phrases that impact as negative behavior on particular groups (as well as containing information reflecting negative behavior which involves physical actions such as denial of service). The use of a machine learning module comprising a search engine designed to monitor language use societally via the Internet enables the methods and systems described herein to be effective in identifying negative behavior and reducing such behavior in real time as social mores change.

The search capabilities embedded in the machine learning module mean that the assessment of negative behavior enabled by the behavior point matrix is based on a level of understanding of language usage societally in a manner that is beyond the capacity of any individual human mind to achieve. No individual human mind can at any given moment know and cross-reference the current frequencies of usage of terms or incidences of particular negative behaviors on multiple heavily trafficked websites or as reported on national and local media websites. Thus, a significant benefit of the methods and systems described herein is that they provide policy makers and concerned organizations and individuals a way to quantify and learn the incidence and distribution of particular negative behaviors societally. Without such accurate and timely information provided by the methods and systems described herein, policy makers or business owners may not even be aware of negative behavior that is occurring and damaging the conduct of their government institutions or corporations. Therefore, the methods and systems described herein enable a significant improvement in the ability to assess and reduce negative behaviors in governmental and corporate contexts.

In some embodiments, the method further comprises the step of generating advice and/or description of remedial measures for the negative behavior/act, and providing feedback to the entity that committed the negative behavior/act. Such feedback could include advice on how to modify their behavior or procedures or even certain objects or other forms of potentially offensive communication in order to minimize future harmful interactions and in the future present a more positive welcoming image to its minority patrons or visitors or participants. This approach could be particularly helpful to well-meaning individuals whose offenses were totally unintentional. By eliminating this ignorance, such people can with modest training learn to behave much more positively. Such advice could be directly targeted at in individual entity or it could be more broadly communicated so that anyone interested in ensuring proper behavior could learn its lessons.

In some embodiments, the method further comprises the step of offering advice to offended parties about what they might do to decrease their odds of encountering discriminatory and/or offensive behavior in the future.

In certain embodiments, the machine learning module comprises a search engine that monitors and identifies examples of restorative justice via discussion on Internet social media or other websites such as media reports. The machine learning can assess the form of restorative justice that typically occurs in response to particular forms of negative behavior. For example, the search engine may identify the incidence of mediation between parties as a response to certain negative behavior; the machine learning module can then assign a point value that reflects the incidence with which such a mode of restorative justice is used in response to that particular negative behavior. The various point values for different modes of restorative justice can then be ranked and compared to the point values assessed for different kinds of negative behavior. In this way, the machine learning module is able to learn and adapt to changing societal mores as to what constitutes an appropriate response to a particular negative behavior. Therefore, the form of restorative justice recommended by the methods and systems described herein may evolve over time.

For example, it may be at the current time that a sincere apology is considered sufficient in response to a particular negative behavior, e.g., sexism. However, it may be reflected in Internet discussion and media reports that over time a particular negative behavior has come to be considered of greater damage to the individual. In such cases, it may be appropriate that stronger forms of restorative justice may be recommended, e.g., financial restitution, retirement/resignation of responsible individuals, etc. The use of a search engine to monitor Internet response to particular negative behavior means that the machine learning module will be able to respond to changing social mores faster and more effectively than an individual human mind will be able to.

Consequently, the methods and systems herein may recommend modes of restorative justice that are stronger than an individual human mind would consider appropriate based on personal experience, but is actually more appropriate based on evolving societal norms. This is a significant improvement over current responses which are often ineffectual and lack impact due to different individual understandings of the degree of negative effects associated with such behavior. In particular, inter-generational differences in understanding about what constitutes an appropriate response to negative behavior may cause senior leadership and management in government or corporations to fail to recognize the damage done by such behavior. This is especially relevant to government institutions or corporations that target a large youth population who may have different perceptions as to appropriate societal behavior than the often older leadership that sets policy. By recommending a strong initial response to a negative information report that identifies negative behavior, the methods and systems described herein will minimize any reputational damage a government institution or corporation will incur with their users or customers.

Types of restorative justice that may be recommended by the methods and systems described herein may include victim/offender mediation or dialogue, conferencing, peace-making circles, victim assistance and involvement, former offender assistance and involvement, real restitution (e.g., financial restitution), community service, anti-bias training, family group decision making, rehabilitation and arbitration. The methods and systems described herein can recommend one or more of these forms of restorative justice based on the assessed severity of the negative behavior identified by the negative information report. Accordingly, the methods and systems described herein improve human behavior by enabling individuals and organizations to filter for the appropriate form of response to make amends for a particular negative behavior. This ensures that offenders do not cause further issues or exacerbate a problem by a response that is considered inadequate, or even perhaps insulting.

In some embodiments, the method further comprises the steps of tracking over time the relative improvement, the lack of improvement or degradations in the rating of the entity, and reporting, when appropriate, the positive actions taken by the entity who committed the negative behavior/act to promote improved behavior.

In certain embodiments, the methods and systems described herein comprise a machine learning module that comprises a search engine that monitors positive comments and reports for an entity or individual who has been previously the subject of a negative information report identifying negative behavior. The entity or individual may be informed of the frequency and value of these positive comments or reports based on assigning them a point value that can be assessed against the degree of negative behavior previously identified. For example, an individual such as a corporate owner who has made comments that are assessed as negative behavior may receive an assessment over time of the extent to which people now view him or her more positively in light of restorative measures he or she has adopted. By basing such an assessment on the frequency and incidence of reports over time, the methods and systems described herein improve human behavior by enabling offenders to both understand better the negative consequences of their behavior, but also to understand a viable means of making amends.

In certain embodiments, entities or individuals that have been responsible for negative behavior may be given an assessment of the typical expected length of time that is required for public comment and views to be changed of them in light of particular restorative actions that they undertake. This will provide offenders a greater ability to plan effective ways of improving their behavior and re-integrating themselves with society.

In some embodiments, the negative information report is only listed under corresponding category, e.g., race, gender or age. For example, an entity's rating or score on sexism may be vastly different than its rating on some other basis, so each type of offensive behavior can be tracked and reported separately as appropriate.

In some embodiments, the method further comprises the step of collecting and sharing appropriate actions taken by both those who are offended and those who work at the entities under review. Sharing such information from one town or city with others around the country or the world could expedite positive behavior especially if successful actions can be identified, widely communicated, and repeated on a large scale.

In some embodiments, the method further comprises the step of collecting and sharing as appropriate results for multi-facility entities. For example, forty restaurants in one part of the country which are part of a chain may be doing great work, but their four counterparts in another part of the country may be behaving inappropriately. Highlighting these discrepancies could help to expedite improvements since headquarters might add pressure to the offending locations in order to avoid criticism of their brand around the country.

In some embodiments, the method further comprises the step of generating and displaying trend lines of various ratings upon request by a user of the application, so that entities and their potential customers could observe signs of improvement or degradation in their behavior.

In some embodiments, the method further comprises the step of receiving, analyzing and listing feedback from the entity on the reported negative behavior/act. The feedback could include disputes, apologies, promises of improved behavior, requests for more detailed reporting, or other public statements the entity may want to make. In some embodiments, the feedback is reviewed by appropriate parties before being made public.

In some embodiments, the method further comprises the step of displaying an advertisement on the application's website. Income from the advertisement could be used to further develop the application and to maintain the application's web site. Resulting revenue sources for the web site could include: entities with good discrimination scores would be motivated to publicize their results; entities with bad scores could publicize their efforts to improve results; and entities that wanted to attract the patrons they lost due to low ratings may be motivated to offer various behavioral incentives through the web site in order to attract previously negative potential customers who might be willing to give them another chance to demonstrate improved behavior.

In some embodiments, the method allows offending parties to offer behavioral incentives to those who have been offended. If a restaurant or other entity has behaved badly and regrets its actions or wants to win back offended parties, then an apology and free or discounted products and/or services or some other benefit might be offered in the hope that they will be given another chance to exhibit improved behavior. These could include, among other things, offers of discounts, or free products or services, or points, or miles, or other behavioral incentives to offended minorities, or other feedback. Such offers might motivate offended parties to give them another chance at winning their support. All such offers would also represent potential revenue sources for the web site that facilitates them. Other sources of revenue for the web site could include payments by various groups of minorities or other groups who might want to display information helpful to their causes. Whether it is advice for members of the group or advice for those who might offend the group, either way there may be useful information that could be shared for a fee while providing a great service to the audience. Another source of revenue for the web site would be sponsoring or facilitating the ability to keep track of audit teams around the country that can audit results if there is a conflict of opinion on any issue. It a store thinks it is behaving properly but it is receiving bad ratings, then it is possible that the ratings are somehow wrong. For example, this could happen if a competitor tried to sabotage the results. For a fee, the web site could sponsor properly vetted individuals who could secretly visit the establishment and give their independent viewpoints.

A behavioral incentive database is stored on a tangible medium accessible by one or more computer processors as part of the system described herein. The behavioral incentive database may comprise both incentives for more positive behavior and incentives offered to atone for negative behavior. The behavioral incentive database may also incorporate behavioral incentives generated by a restorative justice protocol. A restorative justice protocol determines based on the behavioral value of the negative behavior in the negative information report what the appropriate form of incentive is to be offered to reconcile individuals who have suffered negative behavior to the perpetrators of such negative behavior and, thus, rehabilitate the perpetrators.

In certain embodiments, a machine learning module comprises a search engine that monitors and identifies the use of incentives by government institutions or corporations to improve behavior. Based on the results obtained by the search engine, the machine learning module is able to assess a point value based on the incidence of certain incentives to address particular forms of behavior. The different forms of incentive can then be rank ordered and compared to the negative point values that have been assessed for certain forms of behavior. In this manner, the methods and systems described herein are able to incorporate and promote best practices in incentivizing improved behavior at the current time and also evolve and learn what incentives appear to be working best. This is an advantage over current methods since the efficacy of certain incentives may increase or diminish over time in ways that an individual human mind that is limited by personal experience cannot understand. Thus, the behavioral incentive database and its application by the methods and systems described herein acts as a filter to ensure that effective incentives that are appropriate to change particular behaviors are adopted.

In some embodiments, the method further comprises the step of generating, accumulating and publishing strategies for various minorities to employ in order to reduce their likelihood of experiencing discrimination. In certain embodiments, a machine learning module comprises a search engine that monitors discussion of negative stereotypes and the particular behaviors associated with those negative stereotypes. By providing information regarding negative stereotypes to members of minorities, those minority individuals will be better able to assess whether somebody is treating on the basis of a negative stereotype and choose whether to either make a negative information report or to address the individual themselves. This provides a significant advantage over present circumstances by enabling a more accurate assessment of whether negative behavior is based on lack of awareness on the part of an offender or whether such behavior is deliberately calculated to cause harm.

For example, an individual may engage in negative behavior based on a negative stereotype that the individual may not understand that they have developed based on news reports and/or a lack of experience with a particular minority. By raising awareness of the use and type of negative stereotypes that individuals may be exposed to from news reports, Internet discussions, etc., both those individuals engaging in negative behavior and members of minorities may be more quickly able to resolve disputes or identify an inadvertent cause of harm.

In certain embodiments, the behavioral incentive database contains information regarding restorative justice. In other embodiments, the behavioral incentive database is separate from a restorative justice database, which contains information regarding appropriate modes of restorative justice. This information may include, but is not limited to, reporting of business that ceases to exist, possibly due to their loss of customers as a result of the business entity's bad behavior. Entities and individuals can be provided with plans that combine appropriate behavioral incentives and measures for restorative justice. This enhances the ability of offenders to plan a path by which they can improve personal and/or corporate behavior while also make amends for harm caused by negative behavior. By providing a plan for behavioral incentives, restorative justice, as well as in some embodiments a suggested timeline over which an entity or individual should expect to act to make amends, the methods and systems described herein avoid the guesswork is currently involved in such situations. The use of the behavioral point matrix and a machine learning module (with various Internet search capacities) as described herein ensures that the method and systems described herein provide an improvement over current methods of altering human behavior which are over-reliant on the personal feelings and experience of typically older generational leadership in organizations.

In some embodiments, the method further comprises the step of compiling and displaying a list of people who have committed to no longer associate with the offending entity. This could help to ensure the broadest possible reaction to bad behavior if undecided people see that those they respect have decided to stay away.

In some embodiments, the method comprises the step of generating a negative information report database that is accessible to the public. The database may contain information compiled from the negative information reports as well as information from other sources. In some embodiments, the database is easily accessible by all possible devices and methods, such as computers, PC's, laptops, tablets, smart phones, connected televisions, and other display devices. The data could be available by phone, through printed materials, via radio or television broadcasts, movies, or any other form of public or private communication.

The Internet provides a basis for large scale implementation of the method of the present application. The method identifies entities or groups which harbor people who behave poorly and publicize their bad behavior in the hope that behavior will improve or that those offended by it can go elsewhere. This could be a restaurant or a store or a church or a university, or any group of people or entity providing a product or service or meeting opportunity, among other entities. If responsible people identify bad behavior and report it to a central authority, then the data could be accumulated, and feedback could be provided to the entity being criticized and to potential patrons of that establishment. The entity could choose to train its employees to behave better or it could fire discriminating employees or it could ignore the results. Failure to improve behavior could have major financial implications because the resulting bad publicity by the reporting web site and those who view it would greatly reduce the number of people who would want to visit or in any way be associated with the failing entity.

Potential patrons could watch for and report on improvements in behavior or they could avoid the establishment pending future reports of improvements. With appropriate publicity, offended minorities would be motivated to avoid such establishments, but also majorities would often join in the critique and decide to boycott restaurants and stores and other entities or people that do not yet show signs of improvement. Minorities in one category may also support those in other categories. When they are added to the majority people who are also offended by discrimination, the establishment may find that most of its business starts to disappear. For example, if 10% of its customers were black, and 10% were Hispanic, and 10% were gay or lesbian, and 50% were offended by the fact that anyone is subject to discrimination of any sort, that means up to 80% of the customers could decide to take action. Any business or organization that suddenly found 80% of its patrons abandoning it would likely have to close its doors unless it quickly acted to improve its ratings.

In order to get the widest possible coverage of the information available on the discrimination data base, it will need to be easily accessed by all possible devices and methods. These include, but are not limited to, computers, PC's, laptops, tablets, smart phones, connected televisions, and other display devices. The data could be available by phone, through printed materials, via radio or television broadcasts, movies, or any other form of public or private communication.

### System for Implementing the Method

Another aspect of the present application relates to a system for implementing the method of the present application. In some embodiments, the system manages one or more web sites that would highlight local and national stores, restaurants, educational facilities, and other entities or groups of individuals where people might encounter discrimination.

In some embodiments, the system comprises: (i) one or more computing devices in data communication with each other, each device having one or more computer processors, a data communication connection, and one or more tangible non-transitory computer-readable media accessible by the one or more computer processors, and (ii) a plurality of databases, including a verification database, a negative information database, and a behavioral incentive database, wherein the verification database, negative information database and behavioral incentive database are each stored in the one or more tangible non-transitory computer-readable media.

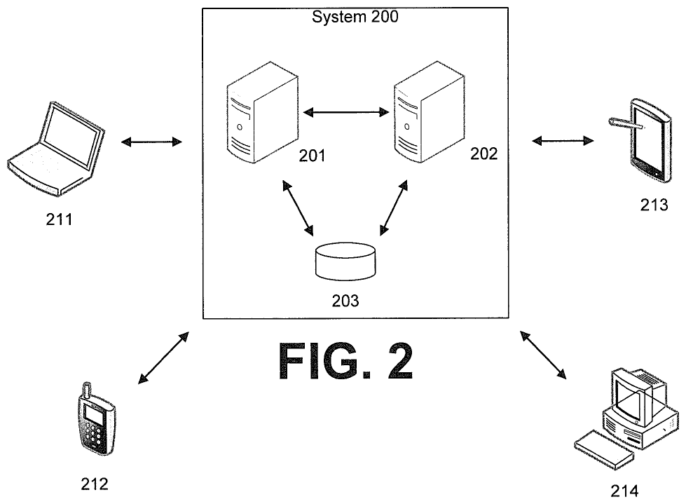

FIG. 2 shows one embodiment of a system 200 used to implement the evaluation/reporting method described above. The system 200 may be hosted on a server 201 or series of servers 201, and these servers may be dedicated or cloud-based. The system 200 may be replicated in embodiments. Embodiments may include various other systems similarly configured and may include and execute one or more subsystem components to perform functions described herein. The system 200 may support one or more databases 203.

The user devices 211, 212, 213, 214 may create data connections with servers 201 using wired, wireless, or network connections. Through these connections, the users can search for records of discriminatory acts or offensive behaviors by an entity of interest, report a discriminatory act or offensive behavior, receive feedbacks from the computer system or from the entity being accused of a discriminatory act or offensive behavior, and execute any other functionality of the system 200. The user devices may be a desktop computer, a laptop computer, a tablet computer, a smartphone, a server, a personal digital assistant, a palmtop computer, or other portable computing device, or any combination of these devices capable of supporting a web browser or other type of application for interacting with the system. Alternatively, the user devices may be limited to a display device capable of displaying output without data processing and memory capabilities. Multiple user devices may be employed either separately or jointly, and a data communication between the devices can be implemented using wired, wireless, or network connections.

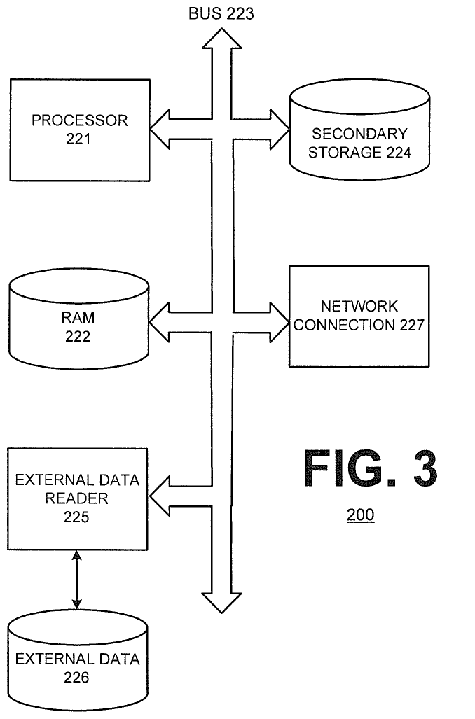

As another embodiment, the system 200 may be implemented locally. Any user devices described in FIG. 2, including a desktop computer, laptop computer, tablet, or mobile device, is suitable to support a local implementation. It is to be understood that the local device may have internet connectivity, and accordingly may have access to cloud-based and server-based system resources. A detailed description of an implemented system 200 is set forth with reference to FIG. 3.

FIG. 3 shows a detailed view of an embodiment of system 200. In this embodiment, the system 200 includes a processor 221, for executing software instructions. Processor 221 may be a single processor or multiple processors, configured as, for example, a bladed server or other known server configurations. Instructions for execution by the processor 221 may be stored in the random access memory (RAM) 222, accessible by the processor 221 through bus 223, in secondary storage 224, in an external data source 226 accessible via external data reader 225, or received from the Internet or other network via network connection 227. The processing by processor 221 may be implemented in software, such as software modules, for execution by computers or other machines. Processor 221 may execute one or more computer programs in order to provide the functions described in this specification. These computer programs preferably include instructions executable to perform the system functions and methods described above and illustrated in the figures herein. Such methods and the processing may be implemented in software, such as software modules, for execution by computers or other machines. The applications preferably provide graphical user interfaces (GUIs) through which users may view and interact with the system. The GUIs may be formatted, for example, as web pages in HyperText Markup Language (HTML), Extensible Markup Language (XML) or in any other suitable form for presentation on a display device depending upon applications used by users to interact with the system 200.

Secondary storage 224 may include a hard disk drive, floppy disk drive, optical disk drive, or other types of non-volatile data storage, and the system 200 may include an external data reader 225 for external data 226, which may be any removable data medium, used if necessary. These components may be interconnected with processor 221 via bus 223 as well. The system 200 may connect with a network, for example, the Internet, or other network, through network connection 227. This connectivity allows for communication with client devices or other computer systems to monitor and harvest data sources, receive user input, or communicate with other systems supporting the system 200. Network connection 207 may accomplish this connection through the use of wired, wireless, or network connections.

In this embodiment, a user device, such as user devices 211, 212, 213, 214 shown in FIG. 2, contains sufficient input/output functionality to support the display of information to a user and that user's interaction with the model. Suitable input devices include a touch-screen, keyboard, mouse, cursor-control device, touch-screen, microphone, digital camera, video recorder or camcorder, and may be used to enter information into GUIs during the user of the system 200. Suitable output devices include any type of device for presenting visual information, such as, for example, a computer monitor or flat-screen display (or mobile device screen), and may include any other device for presenting output, including specialized devices to accommodate sensory-impaired users.

The system 200 may store one or more database structures in secondary storage 224, for example, for storing and maintaining one or more databases, such as database 203 shown in FIG. 2, and other information necessary to perform the methods described herein. Alternatively, the system may include in-memory databases stored in RAM 222 or a database in external data source 226 accessible via data reader 225. Suitable databases include relational, non-relational, transactional, hierarchical, multi-dimensional (e.g., OLAP), object-oriented databases, and the like.

Examples of the system 200 include one or more dedicated server computers, such as bladed servers, personal computers, laptop computers, notebook computers, palm top computers, network computers, mobile devices, or any processor-controlled device capable of executing a web browser or other type of application for interacting with the system. Although only one system 200 is shown in detail, system implementations may use multiple computer system or servers as necessary or desired to support the users and may also use back-up or redundant servers to prevent network downtime in the event of a failure of a particular server. In addition, although system 200 is depicted with various components, one skilled in the art will appreciate that the server can contain additional or different components. In addition, although aspects of an implementation consistent with the above are described as being stored in memory, one skilled in the art will appreciate that these aspects can also be stored on or read from other types of computer program products or computer-readable media, such as secondary storage devices. The computer-readable media may include instructions for controlling system 200 to perform a particular method, such as methods described above.

The computing environment of the present disclosure may include any number of computer or other processing systems (e.g., end-user systems, server systems, etc.) and databases or other repositories arranged in any desired fashion, where embodiments of the present disclosure may be applied to any desired type of computing environment (e.g., cloud computing, client-server, network computing, mainframe, stand-alone systems, etc.). The computer or other processing systems employed by the present disclosure may be implemented by any number of any personal or other type of computer or processing system (e.g., desktop, laptop, PDA, mobile devices, etc.), and may include any commercially available operating system and any combination of commercially available and custom software (e.g., browser software, communications software, server software, etc.). These systems may include any types of monitors and input devices (e.g., keyboard, mouse, head mounted displays, voice recognition, etc.) to enter and/or view information.

Cloud computing may provide internet-based computing, whereby shared servers provide resources, software, and data to computers and other devices on demand. For example, the cloud may be a cloud computing service that includes at least one server computing device, which may include a service abstraction layer and a hypertext transfer protocol wrapper over a server virtual machine instantiated thereon. The server computing device may be configured to parse HTTP requests and send HTTP responses.

Cloud computing uses the Internet and central remote servers to maintain data and applications. A cloud computing service may include a cloud server and cloud storage in communication with a portal e.g., web portal for receiving and transmitting content. Cloud computing can allow users to access and use applications without installation and access personal files at any computer with internet access. Cloud computing can allow for more efficient computing by centralizing storage, memory, processing and bandwidth. The cloud can provide scalable, on-demand computing power, storage, and bandwidth. The cloud storage can be a model of networked computer data storage where data is stored on multiple virtual servers, generally hosted by third parties.

It is to be understood that the software of the present disclosure may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present disclosure may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the embodiments of the present disclosure may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present disclosure may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present disclosure may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (“LAN”), hardwire, wireless link, Intranet, etc.).

The system 200 may employ any number of any conventional or other databases, data stores or storage structures (e.g., files, databases, data structures, data or other repositories, etc.) to store information. The database system may be implemented by any number of any conventional or other databases, data stores or storage structures (e.g., files, databases, data structures, data or other repositories, etc.) to store information. The database system may be included within or coupled to the server and/or client systems. The database systems and/or storage structures may be remote from or local to the computer or other processing systems, and may store any desired data.

In some embodiments, the system 200 contains a machine learning module for maintaining and updating the behavior point matrix.

The embodiments of the present disclosure may employ any number of any type of user interface (e.g., Graphical User Interface (“GUI”), command-line, prompt, etc.) for obtaining or providing information, where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the invention in the form disclosed. Many modifications and variations will be apparent to those of ordinary skill in the art without departing from the scope and spirit of the invention. The exemplary embodiments described herein are presented to explain the principles of the invention and the practical application, and to enable others of ordinary skill in the art to understand the invention for various embodiments with various modifications as are suited to the particular use contemplated.

The terminology used herein was chosen to best explain the principles of the embodiments, the practical application or technical improvement over technologies found in the marketplace, or to enable others of ordinary skill in the art to understand the embodiments disclosed herein; it is to be understood that alternative terminology may be preferred and, therefore, it is the substance of the description, not the semantics, that should control the understanding of the invention.

The present disclosure concerns systems and/or computer program products. A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The computer program products may include a computer readable storage medium (or media) having computer readable program instructions thereon for causing a processor to carry out aspects of the present disclosure. More specifically, the processes and logic flows described in this disclosure can be performed by one or more programmable processors executing one or more computer programs to perform functions by operating on input data and generating output. The processes and logic flows can also be performed by, and apparatus can also be implemented as, special purpose logic circuitry, e.g., a field programmable gate array (“FPGA”) or an application specific integrated circuit (“ASIC”).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a global positioning system (“GPS”) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

In some embodiment, the reports of discriminatory acts or offensive behaviors are entered into the system 200 via the user terminals. The processor 221 verifies the reports based on data stored in the RAM 222 and the secondary storage 224, or based on external data 226 or through network connections 227. The processor 221 then analyzes the report and assigns a total negative information point value to the entity involved in the report based on behavior point matrix generated and updated continually by the processor 221. In some embodiments, the behavior point matrix is generated and maintained by machine learning using cloud-based databases and/or databases stored in the secondary storage 224. In some embodiments, the behavior point matrix is stored in the secondary storage and contains a list of behavioral context categories (e.g., lodging, food, entertainment, medical/dental, legal, etc.). Each category further contains a list of negative behavioral acts (e.g., verbal abuse, physical abuse, material abuse, etc.). A point value is assigned to each negative behavioral act in each behavioral context category. In some embodiments, several point values are assigned to each negative behavior or act in each behavioral context category based on the severity level of the negative behavior or act.

In some embodiments, the system 200 has the capability of being flexible in how data is displayed and how users can view and/or manipulate it. In some versions, local data might be displayed. In other versions, regional or national or global data might be displayed. This would include the entities being evaluated as well as the advertising being offered. Flexibility will add to the value of the solution, but it may be critical that one trusted entity have control over the entire system. This will help to ensure uniform treatment of the many various situations that may arise and will enable the oversight group to ensure that all minorities are protected, not just the most common ones.

In some embodiments, the system further comprises: (i) one or more computing devices in data communication with each other, each device having one or more computer processors, a data communication connection, and one or more tangible non-transitory computer-readable media accessible by the one or more computer processors, and (ii) a plurality of databases, including a verification database, a negative information database, and a behavioral incentive database, wherein the verification database, negative information database and behavioral incentive database are each stored in the one or more tangible non-transitory computer-readable media. In other embodiments, the behavioral incentive database comprises information regarding behavioral incentives and information regarding restorative justice. In other embodiments, the plurality of databases further comprises a restorative justice database.

### Computer Readable Medium

Another aspect of the present application relates to computer readable media, comprising instructions that, when executed by a computer processor, cause the processor to perform: receiving, via a user interface of an application executing on one or more computer processors, a negative information report about an entity, verifying, via the one or more computer processors, the negative information report, assigning, via the one or more computer processors, a severity level point value to each discriminatory act or offensive behavior described in the negative information report based on severity level of the reporter's descriptions and a behavior point matrix stored on a memory device accessible by the one or more computer processors, notifying the entity about the negative information report, and listing the report on a website that is accessible to public if the report is verified in the verifying step.

The computer readable storage medium can be a tangible, non-transitory device that can retain and store instructions for use by an instruction execution device. The computer readable storage medium may be, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (EPROM or Flash memory), a static random access memory (“SRAM”), a portable compact disc read-only memory (“CD-ROM), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network (“LAN”), a wide area network (“WAN”), a cloud computing system and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet using an Internet Service Provider).

Aspects of the present disclosure are described herein with reference to flowchart illustrations and/or block diagrams of methods, apparatus (systems), and computer program products according to embodiments of the invention. It should be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In some embodiments, the tangible, non-transitory computer readable medium comprises instructions that, when executed by a computer processor, cause the processor to receive the negative information report in audio and video form.

In some embodiments, the tangible, non-transitory computer readable medium comprises instructions that, when executed by a computer processor, cause the processor to verify the negative information report using data stored in the system 200 or using external data or through network connections. In some embodiments, the reporter's personal profile passes the verification if 2, 3 or 4 items, such as name, home address, phone number and/or e-mail address, if the reporter's personal profile is confirmed. In some embodiments, the verifying step includes the sub-step of analyzing accumulated data to determine the credibility of those who reported the discriminatory act or offensive behavior (e.g., to avoid having a store's competitor provide distorting results). In some embodiments, the verifying step includes cross checking reports by accredited individuals who may be assigned by one or more recognized authorities to test the entity's behavior. Input from such accredited individuals could be used to clarify any disputed behavior that may have been reported by random individuals. This would be especially important if reports are alleged to be misleading or fraudulent by entities fearing their competitors might be providing false reports.

In some embodiments, the tangible, non-transitory computer readable medium comprises instructions that, when executed by a computer processor, cause the processor to assign the reported act to a defined category of negative behavior (i.e., racism, sexism, anti-gay, etc.) and determines a negative information point value for the reported act based on a behavior point matrix stored in the system 200.

In some embodiments, the tangible, non-transitory computer readable medium comprises instructions that, when executed by a computer processor, cause the processor to update the behavior point matrix regularly, by evaluating a large scale data base reflecting discriminatory acts and/or offensive behaviors described by other reporters, as well as discriminatory acts or offensive behaviors described in other sources, e.g., newspapers, internet, blogs, twitter, etc. In some embodiments, the behavior point matrix contains a list of behavioral context categories (e.g., lodging, food, entertainment, medical/dental, legal, etc.). Each category further contains a list of negative behavioral acts (e.g., verbal abuse, physical abuse, material abuse etc.). The point value assigned to each negative behavioral act in each behavioral context category, as well as point values reflecting severity level of the negative behavioral act in each behavioral context category, may be adjusted from time to time.

In some embodiments, the tangible, non-transitory computer readable medium comprises instructions that, when executed by a computer processor, cause the processor to generate the matrix using machine learning and a large database of negative behavioral acts and related events. In some embodiments, the tangible computer readable medium comprises instructions that, when executed by a computer processor, cause the processor to analyze many types of information derived from the negative information report, such as verbal expression patterns, sentence structure, key words, gestures, body movement, environment, personal information (e.g., race, gender, religion, age, etc.) of the person who committed the discriminatory act or offensive behavior, personal information of the person being adversely affected by the discriminatory act or offensive behavior. The device may contain one or more memory units record several types of information. Examples of the types are the patterns which express the information, connective information with the other memory unit expressing relations between the information (the number of connections, address of the connection, a relation with the patterns to connect), conditions to activate (compare and match conditions), and results of the analysis (sources of the information, reliability, newness, fields, theme, types of the sentences, etc.).

In some embodiments, the tangible, non-transitory computer readable medium comprises instructions that, when executed by a computer processor, cause the processor to generate advice and/or descriptions of remedial measures for the discriminatory act or offensive behavior, and providing feedback to the entity which committed the discriminatory act or offensive behavior.

In some embodiments, the tangible, non-transitory computer readable medium comprises instructions that, when executed by a computer processor, cause the processor to track over time the relative improvement or degradations in the rating of the entity.

In some embodiments, the tangible, non-transitory computer readable medium comprises instructions that, when executed by a computer processor, cause the processor to collect and share appropriate actions taken by both those who are offended and those who work at the entities under review.

In some embodiments, the tangible, non-transitory computer readable medium comprises instructions that, when executed by a computer processor, cause the processor to generate and display trend lines of various ratings upon request by a user of the application, so that entities and their potential customers could observe signs of improvement or degradation in their behavior.

In some embodiments, the tangible, non-transitory computer readable medium comprises instructions that, when executed by a computer processor, cause the processor to receive, analyze and list feedback from the entity on the reported discriminatory act or offensive behavior. The feedback could include disputes, apologies, promises of improved behavior, requests for more detailed reporting, or other public statements the entity may want to make. In some embodiments, the feedback is reviewed by appropriate parties before being made public.

In some embodiments, the tangible, non-transitory computer readable medium comprises instructions that, when executed by a computer processor, cause the processor to display an advertisement on the application's website.

The tangible non-transitory computer readable storage medium in some embodiments, further comprises instructions that, when executed by a computer processor, cause the processor to update the behavior point matrix based on monitoring incidences of discriminatory or offensive behaviors using search terms or phrases consistent with the exercise of discriminatory or offensive behaviors. In other embodiments, the tangible computer readable media further comprises instructions that, when executed by the one or more computer processors, cause the one or more computer processors to retrieve, via the one or more computer processors, information about how to incentivize improved behavior from a behavioral incentive database stored on a memory device. In other embodiments, the tangible computer readable media further comprises instructions that, when executed by the one or more computer processors, cause the one or more computer processors to retrieve, via the one or more computer processors, information about restorative justice to resolve harm caused by negative behavior from a restorative justice database stored on a memory device. In other embodiments, the tangible computer readable media further comprises instructions that, when executed by the one or more processors, cause the one or more processors to electronically deliver, via the one or more computer processors, a behavioral report to the individual or entity that is the subject of that report.

One of ordinary skill will understand that the embodiments described herein above do not represent the full range of embodiments encompassed by the methods and systems described herein. The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence which is effective to meet the objectives there intended, unless the context specifically indicates the contrary.

1. A method for monitoring and modifying behavior to reduce negative effects, comprising the steps of:
(a) receiving, via a user interface of an application executing on one or more computer processors, a negative behavior information report about one or more discriminatory acts or offensive behaviors known or alleged to have been committed by an individual or entity, said information being obtained from a private and/or confidential source, a website reporting said discriminatory acts or offensive behaviors news, or both;
(b) assigning, via the one or more computer processors, a negative behavior information point value for any of a plurality of discriminatory acts or offensive behaviors and a discriminatory/offensive behaviors point matrix stored on a memory device accessible by the one or more computer processors, the discriminatory/offensive behaviors point matrix being generated and continually updated by a machine learning module, wherein the machine learning module comprises a learning tool for identifying effective strategies for addressing discriminatory or offensive behaviors, based on effective response strategies identified and processed by the machine learning module;
(c) determining, via the one or more computer processors, a total negative behavior information point value of the individual or entity via the one or more computer processors, based on the sum total of negative behavior information point values in real-time;
(d) storing the negative behavior information report on a negative behavior information database;
(e) notifying the individual or entity about the negative behavior information report and notifying the individual or entity of a proposed response strategy to address one or more discriminatory acts or offensive behaviors alleged to have been committed by the individual or entity, wherein the proposed response is determined by the machine learning module; and
(f) providing a website for reporting negative behavior information reports.

2. The method of claim 1, wherein the negative behavior information report is based on information obtained from a private and/or confidential source.

3. The method of claim 1, wherein the negative behavior information report is based on information obtained from an online news source.

4. The method of claim 1, wherein the machine learning module comprises a search engine that automatically tracks reported incidences of discriminatory acts or-offensive behaviors by the individual or entity in one or more websites.

5. The method of claim 4, wherein the search engine monitors the incidences of discriminatory acts or offensive behaviors using search terms or phrases consistent with the exercise of discriminatory acts or offensive behaviors.

6. (canceled)

7. The method of claim 1, further comprising a step of verifying the authenticity of the negative behavior information report.

8. The method of claim 1, wherein the negative behavior information report is listed on the website in step (f).

9. The method of claim 8, wherein the negative behavior information report is listed on the website in step (f) only after verifying the authenticity of the negative behavior information report.

10. (canceled)

11. The method of claim 1, wherein the negative behavior information report is withheld from being listed on the website in step (f) when the individual or entity has completed the proposed response strategy within a specified time period.

12. The method of claim 1, further comprising the step of listing on the website in step (f) positive actions taken by the individual or entity with respect to one or more negative behavior information reports or positive actions more generally that are worthy of note due to their effects on potential discriminatory actions.

13. The method of claim 1, further comprising the step of tracking over time, via the one or more computer processors and the total negative behavior information point values, the relative improvement or lack of improvement over time with respect to discriminatory acts or offensive behaviors by the individual or entity.

14. The method of claim 1, further comprising the step of providing a system comprising:
(i) one or more computing devices in data communication with each other, each device having one or more computer processors, a data communication connection, and one or more tangible non-transitory computer-readable media accessible by the one or more computer processors, and
(ii) a plurality of databases, including a verification database, a negative information database, and a behavioral incentive database, wherein the verification database, negative information database and behavioral incentive database are each stored in the one or more tangible non-transitory computer-readable media.

15. The method of claim 14, wherein the behavioral incentive database comprises information regarding behavioral incentives and information regarding restorative justice.

16. The method of claim 14, wherein the plurality of databases further comprises a restorative justice database.

17. A system for monitoring and modifying negative behaviors, comprising:
one or more computer processors; and
one or more tangible computer readable media accessible by the one or more computer processors, wherein the one or more tangible computer readable media comprise instructions that, when executed by the one or more processors, cause the one or more processors to perform:
(a) receiving, via a user interface of an application executing on one or more computer processors, a negative behavior information report about one or more discriminatory acts offensive behaviors known or alleged to have been committed by an individual or entity, said information being obtained from a private and/or confidential source, a website reporting said discriminatory acts or offensive behaviors news, or both;
(b) assigning, via the one or more computer processors, a negative behavior information point value for any of a plurality of discriminatory acts or offensive behaviors and a discriminatory/offensive behaviors point matrix stored on a memory device accessible by the one or more computer processors, the discriminatory/offensive behaviors point matrix being generated and continually updated by a machine learning module, wherein the machine learning module comprises a learning tool for identifying effective strategies for addressing discriminatory acts or offensive behaviors, based on effective response strategies identified and processed by the machine learning module;
(c) determining, via the one or more computer processors, a total negative behavior information point value of the individual or entity via the one or more computer processors, based on the sum total of negative behavior information point values in real-time;
(d) storing the negative behavior information report on a negative behavior information database;
(e) notifying the individual or entity about the negative behavior information report and notifying the individual or entity of a proposed response strategy to address one or more discriminatory acts or offensive behaviors alleged to have been committed by the individual or entity, wherein the proposed response is determined by the machine learning module; and
(f) providing a website for reporting negative behavior information reports.

18. The system of claim 17, wherein the negative behavior information report is based on information obtained from a private and/or confidential source.

19. The system of claim 17, wherein the negative behavior information report is based on information obtained from an online news source.

20. The system of claim 17, wherein the machine learning module comprises a search engine that automatically tracks reported incidences of discriminatory acts or offensive behaviors by the individual or entity in one or more websites.

21. The system of claim 20, wherein the search engine monitors the incidences of discriminatory acts or offensive behaviors using search terms or phrases consistent with the exercise of discriminatory acts or offensive behaviors.

22. (canceled)

23. The system of claim 17, wherein the one or more processors verify the authenticity of the negative behavior information report.

24. The system of claim 17, wherein the negative behavior information report is listed on the website in step (f).

25. The system of claim 24, wherein the negative behavior information report is listed on the website in step (f) only after verifying the authenticity of the negative behavior information report.

26. (canceled)

27. The system of claim 17, wherein the one or more processors cause the system to withhold listing of the negative behavior information report on the website in step (f) when the individual or entity has completed the proposed response strategy within a specified time period.

28. The system of claim 17, further comprising the step of listing on the website in step (f) positive actions taken by the individual or entity with respect to one or more negative behavior information reports.

29. The system of claim 17, wherein the one or more computer processors track the total negative behavior information point values over time and report on the website the relative improvement or lack of improvement over time with respect to discriminatory acts or offensive behaviors by the individual or entity.

30. A tangible non-transitory computer readable storage medium, comprising instructions that, when executed by a computer processor, cause the processor to:
(a) receive, via a user interface of an application executing on one or more computer processors, a negative behavior information report about one or more discriminatory acts or offensive behaviors known or alleged to have been committed by an individual or entity, said information being obtained from a private and/or confidential source, a website reporting said discriminatory acts or offensive behaviors news, or both;
(b) assign, via the one or more computer processors, a negative behavior information point value for any of a plurality of discriminatory acts or offensive behaviors and a discriminatory/offensive behaviors point matrix stored on a memory device accessible by the one or more computer processors, the discriminatory/offensive behaviors point matrix being generated and continually updated by a machine learning module, wherein the machine learning module comprises a learning tool for identifying effective strategies for addressing discriminatory acts or offensive behaviors, based on effective response strategies identified and processed by the machine learning module;
(c) determine, via the one or more computer processors, a total negative behavior information point value of the individual or entity via the one or more computer processors, based on the sum total of negative behavior information point values in real-time;
(d) store the negative behavior information report on a negative behavior information database;
(e) notify the individual or entity about the negative behavior info illation report and notifying the individual or entity of a proposed response strategy to address one or more discriminatory acts or offensive behaviors alleged to have been committed by the individual or entity, wherein the proposed response is determined by the machine learning module, and
(f) list the negative behavior information report on a website when certain conditions are met.

31. The method of claim 1, wherein the one or more discriminatory acts or offensive behaviors known or alleged to have been committed by an individual or entity are based on differential treatment of individuals according to one or more status groups selected from the group consisting of: gender, race, age, ethnicity, disability, marital status, sexual orientation, political affiliation, national/geographic origin, religion, language, immigration status, medical status, pregnancy status, criminal record, personal appearance, social class, address or zip code, geographical origin, and economic status.
